(19) United States
(12) Patent Application Publication
(10) Pub. No.: US 2022/0226560 A1
(43) Pub. Date: Jul. 21, 2022

(54) DEVICES, METHODS, AND SYSTEMS FOR IMPROVED ENEMA AND RECTAL IRRIGATION PROCEDURES

(71) Applicant: Elsa GONZALES, Sunny Isles Beach, FL (US)

(72) Inventor: Elsa GONZALES, Sunny Isles Beach, FL (US)

(21) Appl. No.: 17/154,983

(22) Filed: Jan. 21, 2021

(51) Int. Cl. A61M 3/02; A61M 39/28

(52) U.S. Cl. CPC A61M 3/0245; A61M 3/0287; A61M 2205/584; A61M 39/28; A61M 3/0279

(57) ABSTRACT

Methods of administering an enema to a patient utilizing a fecal management system are disclosed. Such methods may include providing the patient utilizing the fecal management system and providing an enema solution providing apparatus. The enema solution providing apparatus may include a bag containing enema solution, tubing, a tubing connector, and an adjustable clamp. The method may include clamping a catheter of the fecal management system between an irrigation port and an collection bag, engaging the tubing connector with the irrigation port, actuating the adjustable clamp to commence a flow from the bag containing enema solution into the patient through the catheter, completing the flow from the bag containing enema solution into the patient, disengaging the tubing connector from the irrigation port; and unclamping the catheter. Kits for providing an enema through a fecal management system and enema solution providing apparatuses are also disclosed.

### TECHNICAL FIELD

This disclosure relates to devices, methods, and systems for providing enemas and rectal irrigation. More particularly, this disclosure relates to improvements to the provision of enemas and rectal irrigation to patients being treated with a fecal management system.

### BACKGROUND

Patients suffering from constipation, other gastro-intestinal maladies, and other conditions are commonly provided with enemas to aid in the removal of waste products from the gastro-intestinal tract and/or administer medication. During an enema, a saline solution, a soap solution, a medicated solution, and/or the like may be anally administered to a patient. In the hospital setting, a medicated solution may be prescribed by a physician and provided by a pharmacy for administration by a nurse or other medical professional. Soap enema solutions may be used to treat fecal impaction, relieve pain, and/or the like. Common medicated enema solutions may include pain relievers, such as acetaminophen or ibuprophen; lactulose for patients suffering from alcohol withdrawal; antibiotics, such as Vancomyacin to treat and prevent bacterial infection (e.g., from C. difficile); combination antibiotics to eradicate protozoan parasites (e.g., B. hominis & D. fragilis); and/or others medicated solutions known in the art.

FIG. 2 depicts a generic traditional enema system 50. Enema system 50 may comprise an enema bag 60 and a catheter 70. Enema bag 60 may include a fill port 65; in other examples, enema bag 60 may be prefilled. Catheter 70 may have insertion end 74 and may be attached to and receive fluid from enema bag 60. Clamp 72 may be positioned along catheter 70 and may be configured to block fluid flow prior to enema administration, stop fluid flow after administration has commenced, and/or the like.

During operation of traditional enema system 50, a filled enema bag 60 is provided and positioned above the patient, typically using an IV pole, hook, hanger, and/or the like. Insertion end 74 is lubricated and then positioned within the patient's rectum. Clamp 72 may then be released to allow enema solution to flow from enema bag 60, through catheter 70, and into the patient.

Traditional enema systems 50 are commonly used, but have many drawbacks. First, the tubing may be thick, typically with an inner diameter of approximately 0.8 cm to 1.5 cm ( 5/16″ to 9/16″, a wider outer diameter, and may comprise hard vinyl. This thicker tubing may cause patient discomfort due to (1) a faster than desirable flow of enema solution through catheter 70 and correspondingly rapid delivery of enema solution into the patient, and (2) a larger insertion end 74 being placed and maintained with the patient's rectum. In some circumstances, commonly used variations of insertion end 74 may be difficult to insert due to impacted stool, swelling, or other blockages or exigencies. Traditional enema systems 50 also have the disadvantage of being messy and unhygienic to use in many circumstances: After the enema is administered, liquid waste products may uncontrollably and rapidly exit the patient's body as soon as, or sometime before, insertion end 74 is removed from the rectum.

Existing fecal management systems 1 (referred to as “FMS”) serve to capture liquid or semi-liquid stool and other waste products from a patient suffering from gastrointestinal issues, typically incontinence, while substantially mitigating and/or preventing messy and unhygienic situations. Commercially available FMS include Flexi-Seal® PROTECT FMS and Flexi-Seal® SIGNAL® FMS sold by ConvaTec Inc., and DIGNISHIELD® Stool Management System sold by C.R. Bard, Inc.

FIG. 1 depicts existing fecal management system 1. FMS 1 may include collection bag 10; catheter 20; syringe 40; and cinch clamp 34, which may positioned on catheter 20 or included separately (as shown). At one end, catheter 20 may include FMS intake 24 which is configured to be inserted into a patient's rectum; at the opposite end, catheter 20 may include catheter expulsion port 22. FMS intake 24 may include an annular retention balloon 28 to help maintain positioning during use, and such retention balloon may be inflated and deflated (typically with a saline solution) via inflation port 26. Catheter 20 may further include an irrigation/medication port 30, which in turn may include an irrigation port connector 32, such as, but not limited to, a female luer lock connector. Irrigation/medication port 30 may be self-sealing such that it remains closed when not in use. Catheter 20 may further include sample port 21 for collecting stool or other specimens for laboratory study. Catheter 20 may further include hanging strap 29 to help secure and position collection bag 10. Collection bag 10 may include bag intake port 12 to engage with catheter expulsion port 22; bag 10 may ultimately to receive waste from catheter 20.

During operation of FMS 1, collection bag 10 may be connected to catheter 20 via catheter expulsion port 22 and bag intake port 12. FMS intake 24 may be inserted into a patient's rectum; retention balloon 28 may be inflated via inflation port 26 to maintain its positioning and create a suitable seal against the internal portion of a patient's rectum. Syringe 40 may be used for such inflation. Using hanging strap 29, collection bag 10 may be secured within the patient's environment so that gravity draws waste into it.

As appropriate, a nurse or other medical professional may maintain safe and effective operation of FMS 1 by a process commonly referred to as irrigation. During irrigation, saline or another fluid may be injected into irrigation/medication port 30 in order to clean the tubing and to reduce the likelihood of obstructions resulting from solid fecal matter or other substances.

As appropriate, a nurse or other medical professional may administer enemas through FMS 1. To administer an FMS enema, a medical professional may first clamp catheter 20 between irrigation/medication port 30 and catheter expulsion port 22 to prevent the enema solution from flowing directly into collection bag 10, and thereby enabling flow of the enema solution into the patient. Typical FMS patient care in, for example, the ICU, may require administration of an enema solution every 6-8 hours via repeated syringe injections into irrigation/medication port 30.

While the above-referenced process may effectively administer an enema to a patient, it has many drawbacks. First, a typical enema for an adult may require administration of approximately 600 ml of enema solution. Existing syringes corresponding the irrigation/medication port 30 of existing fecal management systems are typically 60 ml. Accordingly, each enema administration may require a nurse or other medical professional to both (a) fill the syringe with the appropriate enema solution, and (b) inject the enema solution into irrigation/medication port 30, approximately ten times. A medical professional proficient in administering this repetitious process may spend approximately 30-45 minute or more to complete the process, perhaps with a majority of the time spent refilling the syringe. Less experienced medical professionals may require even more dedicated time to administer an FMS enema. Given the many, varied, and significant demands on nurse time, in the ICU and elsewhere—as well as nurse staffing shortages in many medical facilities—a substantial reduction in FMS enema administration time would be extremely desirable.

Second, FMS enema administration often causes substantial patient discomfort due to the rapid flow of enema solution into a patient's rectum. While a nurse might be able to more slowly inject each syringe of enema solution into irrigation/medication port 30 if a patient complains of discomfort, this would represent a substantial increase the FMS enema administration time, which is already far too long. Additionally, maintaining a patient-desired injection speed may be needlessly cumbersome, difficult, and physically demanding for nurses. This is especially problematic because the existing, repetitive FMS enema process may be typically be required several times a day for a plurality of patients in a given ICU or other medical facility.

Thus, a need exists for improved methods of administering enemas, as well as apparatuses, systems, and kits to support such improved methods.

### SUMMARY

The present disclosure provides a description of apparatuses, systems, kits thereof, and methods of use thereof to address the perceived problems described above.

In one embodiment, a method of administering an enema to a patient utilizing a fecal management system is provided. The fecal management system may include an irrigation port, a catheter, and a collection bag. The method may include providing the patient utilizing the fecal management system and providing an enema solution providing apparatus. The enema solution providing apparatus may include a bag containing enema solution, tubing, a tubing connector, and an adjustable clamp. The method may further include clamping the catheter between the irrigation port and the collection bag, engaging the tubing connector with the irrigation port, actuating the adjustable clamp to commence a flow of enema solution from the bag containing enema solution into the patient through the catheter, completing the flow of enema solution from the bag containing enema solution into the patient, disengaging the tubing connector from the irrigation port, and unclamping the catheter.

The method may further include actuating the adjustable clamp to adjust the flow. This step may occur after the step of actuating the adjustable clamp to commence the flow and before the step of completing the flow.

The enema solution providing apparatus may further include a drip chamber. The method may further include monitoring the drip chamber prior to and/or after the step of actuating the adjustable clamp to adjust the flow.

The step of actuating the adjustable clamp to adjust the flow may include receiving feedback from the patient and actuating the adjustable clamp to adjust the flow in response to the feedback.

The adjustable clamp may be a roller clamp. The step of actuating the adjustable clamp to adjust the flow may include rolling a roller of the roller clamp.

The step of actuating the adjustable clamp to commence the flow of enema solution may further include actuating the adjustable clamp to an intermediate position between blocking flow and allowing maximum flow.

The step of engaging the tubing connector with the irrigation port may further include engaging a female leur lock connector of the irrigation port with male leur lock connector of the tubing connector.

The step of completing the flow of enema solution from the bag containing enema solution into the patient may further include actuating the adjustable clamp to block the flow.

The step of providing the enema solution providing apparatus may further include providing the enema solution providing apparatus with a tubing connector color that corresponds to an irrigation port color.

In another embodiment, a kit for providing an enema through a fecal management system is provided. The kit may include an adjustable clamp, tubing, and a tubing connector. The tubing may be configured to attach to a drip bag at a first end. The tubing connector may be provided at a second end of the tubing.

The tubing connector may include a male leur lock connector.

The adjustable clamp may be a roller clamp.

The kit may further include a drip chamber. The drip chamber may be provided along the path of the tubing.

The kit may further include the drip bag. The drip bag may be pre-filled with an enema solution.

The kit may further include a fecal management system.

In yet another embodiment, an enema solution providing apparatus is provided. The apparatus may include a drip bag, tubing, an adjustable clamp, and a tubing connector. The drip bag may contain an enema solution. The tubing may be fluidly connected to the drip bag at a first end. The adjustable clamp may be positioned along the tubing. The tubing connector may be attached to a second end of the tubing and may include a male leur lock connector. The adjustable clamp may have at least a first position configured to block a flow of the enema solution through the tubing, a second position configured to allow a maximum flow of the enema solution through the tubing, and a third position configured to allow an intermediate flow of the enema solution through the tubing.

The adjustable clamp may be a roller clamp.

The apparatus may further include a drip chamber provided along the path of the tubing.

### BRIEF DESCRIPTION OF THE DRAWINGS

The accompanying drawings, which are incorporated into and constitute a part of this disclosure, illustrate several embodiments and aspects of the compositions, and methods described herein and, together with the description, serve to explain the principles of the invention.

FIG. 1 is an illustration of an existing fecal management system.

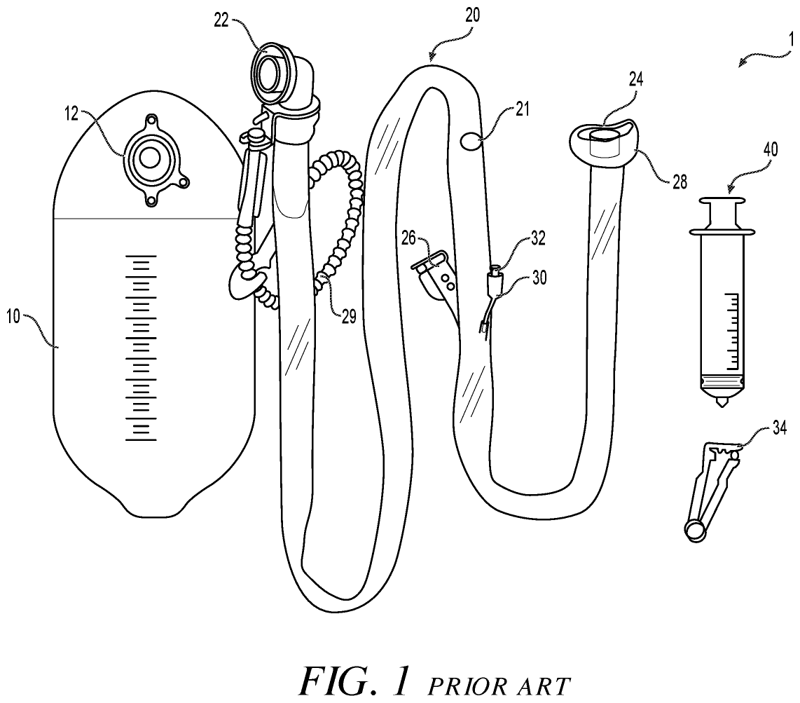

FIG. 2 is an illustration of a traditional enema system.

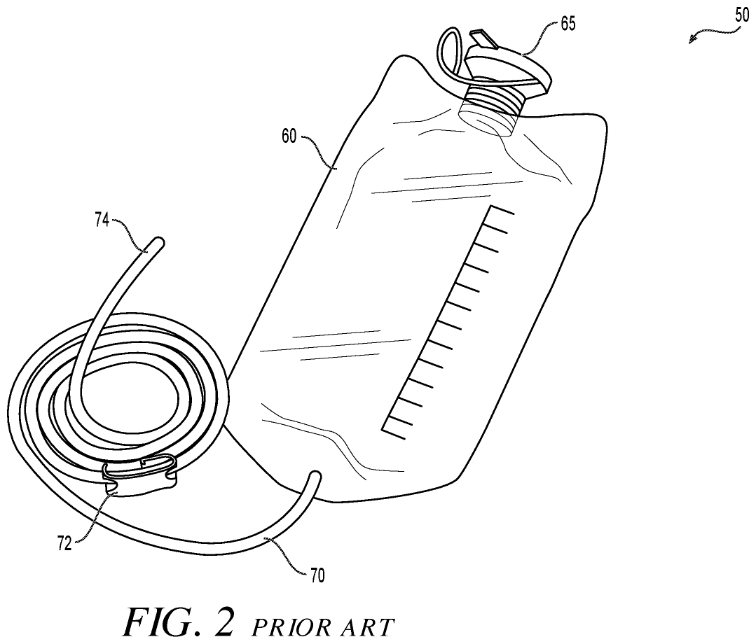

FIG. 3 is an illustration of an enema solution providing apparatus, in accordance with exemplary embodiments.

FIG. 4 is an illustration of an enema administration device, in accordance with exemplary embodiments.

FIG. 5 is a flowchart of a method of administering an enema, in accordance with exemplary embodiments.

FIGS. 6A and 6B are side and top illustrations, respectively, of an insertion tip, in accordance with exemplary embodiments.

### DETAILED DESCRIPTION

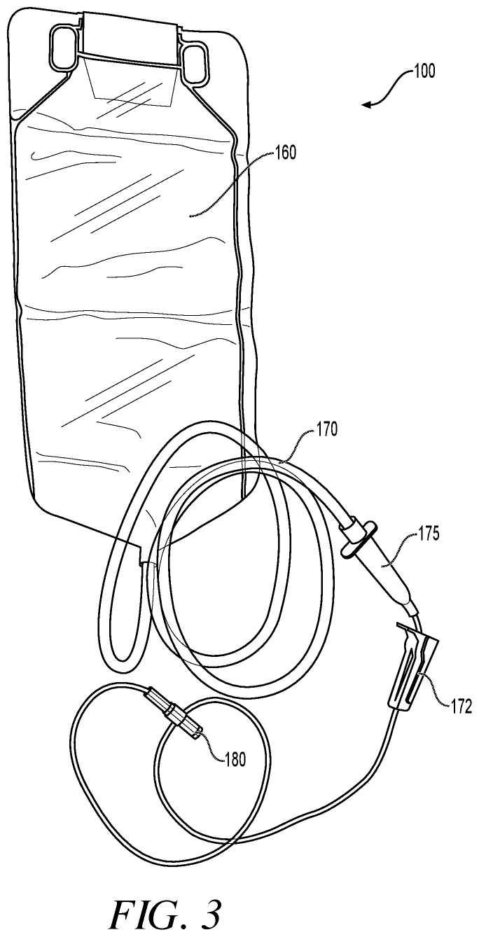

With reference to FIG. 3, an exemplary embodiment of an enema solution providing apparatus 100 is disclosed. Apparatus 100 may comprise drip bag 160, tubing 170, and adjustable clamp 172. Tubing 170 may be attached to drip bag 160. Apparatus 100 may further comprise tubing connector 180 at the opposite end of tubing 170 from drip bag 160. Apparatus 100 may further comprise drip chamber 175 provided along tubing 170 to provide a visual indication of fluid flow therethrough. Drip bag 160 may be a common IV drip bag, an enema bag, or any other suitable liquid storage device known in the art. In some embodiments, drip bag 160 may comprise silicone or vinyl.

In some embodiments, tubing 170 may have an inner diameter of 0.8 cm to 1 cm, but this disclosure is not so limited. In other embodiments, for example as shown in FIG. 3, tubing 170 may have an upper tubing component (e.g., provided between drip bag 160 and drip chamber 175) and a lower tubing component (e.g., provided between drip chamber 175 and tubing connector 180). In some embodiments, upper tubing component may have a diameter of 0.8 cm-1 cm and lower tubing component may have a diameter of 0.3 cm-0.5 cm.

It is contemplated that tubing connector 180 may be configured to fluidly connect with irrigation/medication port 30 of an existing fecal management system 1. Because in existing fecal management systems 1, irrigation port connector 32 is typically a female leur lock connector, tubing connector 180 may be a male leur lock connector in preferred embodiments. Additionally, it is contemplated that tubing connector 180 may substantially match irrigation port connector 32 in color to avoid potential medical provider confusion—which may in turn result in wasted medical provider time and effort; wasted medical equipment; more seriously, potentially dangerous fecal or other contamination; and/or the like. Port 30 of the DIGNISHIELD® Stool Management System is generally white or translucent in color. Accordingly, it is contemplated that tubing connector 180 may be substantially white and/or translucent in some embodiments. Port 30 of the Flexi-Seal® PROTECT FMS and Flexi-Seal® SIGNAL® FMS are generally light blue in color. Accordingly, it is contemplated that tubing connector 180 may be substantially blue in some embodiments. In other embodiments, teal, green, turquoise, or other colors that “pop” may be utilized, particularly if they match or somewhat resemble the irrigation port 30 color

In certain embodiments, adjustable clamp 172 may be a roller clamp; it may be provided on tubing 172. In alternative embodiments, adjustable clamp 172 may be any device known in the art with at least three positions: (1) fully blocking flow through tubing 170, (2) substantially allowing flow through tubing 170, and (3) at least one intermediate position where flow is substantially reduced.

It is contemplated that enema solution providing apparatus 100 may be utilized in conjunction with an existing fecal management system 1 to administer enemas to a patient and/or irrigate an existing fecal management system 1 in a substantially improved manner. That is, utilization of apparatus 100 may result in a substantial savings of medical personnel time and effort and/or may simultaneously reduce patient discomfort.

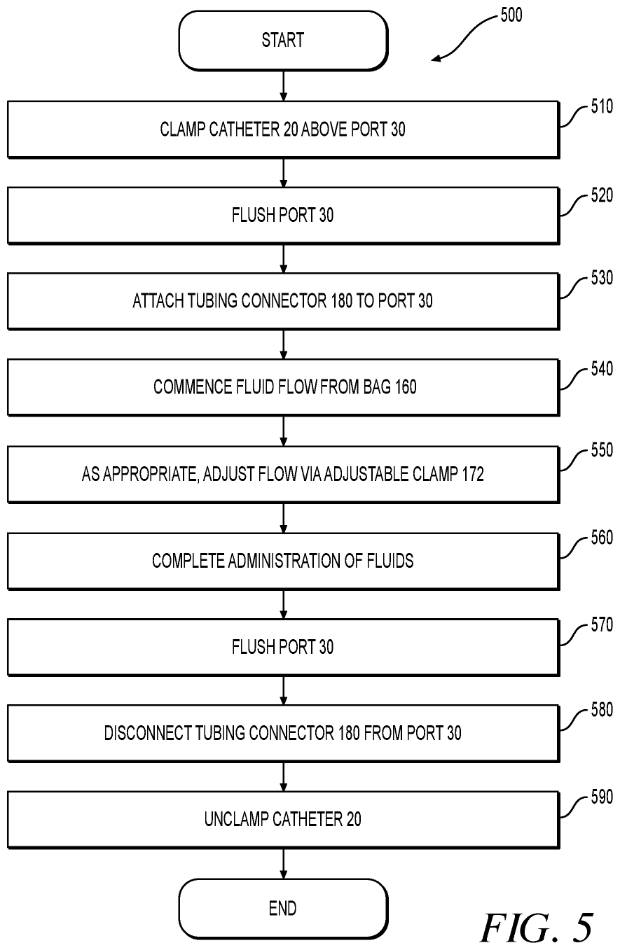

With reference to FIG. 5, an exemplary method 500 for administering an enema with enema solution providing apparatus 100 is disclosed. It is contemplated that method 500 may proceed on a patient who is already utilizing fecal management system 1. Apparatus 100 may be provided, and a filled drip bag 160 may be positioned above the patient, using, for example, an IV pole, hook, hanger, and/or the like.

As in step 510, catheter 20 may be blocked between irrigation/medication port 30 and catheter expulsion port 22 to prevent the enema solution from flowing directly into collection bag 10. In some embodiments, cinch clamp 34 may be used. The process may proceed to step 510.

As in step 520, irrigation/medication port 30 may be flushed. For example, approximately 60 ml of saline may be injected into irrigation/medication port 30 via syringe 40 or another syringe. Step 520 may be omitted in some embodiments. The process may proceed to step 530.

As in step 530, tubing connector 180 of apparatus 100 may be connected to irrigation/medication port 30. This may be accomplished by engaging irrigation port connector 32 with tubing connector 180. The process may proceed to step 540.

As in step 540, adjustable clamp 172 may be actuated such that tubing 170 is no longer blocked. Where clamp 172 is a roller clamp, its roller may be rolled to initiate the flow of enema solution. Accordingly, the enema solution in drip bag 160 may begin to flow through tubing 170; into catheter 20 via irrigation/medication port 30; and into the patient via FMS patient intake port 24. The flow of enema solution may be confirmed or assessed by observing drip chamber 175. The process may proceed to step 550.

As in step 550, the nurse or medical practitioner may regulate the flow of enema solution by actuating adjustable clamp 172. The flow of enema solution may be assessed by observing drip chamber 175. Flow may be regulated based upon, for example, patient reports of discomfort, physician instructions, a patient's medical condition, and/or prior experiences in administering enemas to the patient or similarly situated patients. In some embodiments, adjustable clamp 172 may be adjusted to an intermediate position between blocking flow and allowing maximum flow. The process may proceed to step 560.

As in step 560, the administration of enema solution may be permitted to complete. In some embodiments, the rate of flow of enema solution may be re-regulated, as in step 550, one or multiple times during step 560. In some embodiments, the administration of enema solution may continue until drip bag 160 is substantially emptied; in other embodiments, step 560 may be concluded earlier by closing clamp 172 and blocking flow.

It is contemplated that, in various embodiments, administration of enema solution may range from approximately five minutes to approximately an hour, depending on medical circumstances, including but not limited to physician instructions and the patient's subjective discomfort level. For example, it may be advantageous for a patient receiving a medicated enema, such as an antibiotic enema, to receive a slow rate of flow so that the medication has sufficient time to be effective and/or to minimize patient discomfort. In other circumstances, medical exigencies may advocate for faster flow.

In some embodiments, a typical administration time may be between 10 and 35 minutes, or more preferably between 15 and 30 minutes. Such times are similar to prior art FMS 1 syringe-based enema administration times discussed above. However, when apparatus 100 is utilized, the nurse can tend to other patients and engage in other activities while the enema solution is being administered. That is, it is contemplated that after the flow rate is adjusted as in step 550, the patient may need only passive monitoring until step 560 is completed. Accordingly, substantial savings in medical personnel resources may be realized by utilizing apparatus 100 and/or method 500.

Furthermore, even where the total fluid provision time is the same, administering an FMS enema utilizing apparatus 100 may be characterized by relatively stable and slower flow speeds. By contrast, administering an FMS enema without apparatus 100 may be characterized by starts and stop—and faster intermittent flow speeds—to achieve the same total fluid provision time.

While in preferred embodiments, the flow of enema solution out of drip bag 160 may be driven by gravity, other modes of fluid provision are contemplated. For example, an infusion pump or other fluid administration mechanism known in the art may be used. In such embodiments, adjustable clamp 172 may be replaced or modified as appropriate.

The process may proceed to step 570.

As in step 570, irrigation/medication port 30 may be flushed. For example, approximately 60 ml of saline may be injected into irrigation/medication port 30 via syringe 40 or another syringe. Step 570 may be omitted in some embodiments. The process may proceed to step 580.

As in step 580, tubing connector 180 may be disconnected from irrigation/medication port 30. Apparatus 100 may be discarded. The process may proceed to step 590.

As in step 590, the blockage of catheter 20 between irrigation/medication port 30 and catheter expulsion port 22 may be removed. For example, where cinch clamp 34 was utilized to block flow, it may be released. Removing the blockage will allow the used enema solution, along with waste products from the patient to flow into collection bag 10. Method 500 may be completed.

A method of irrigation may proceed in a substantially similar manner as method 500.

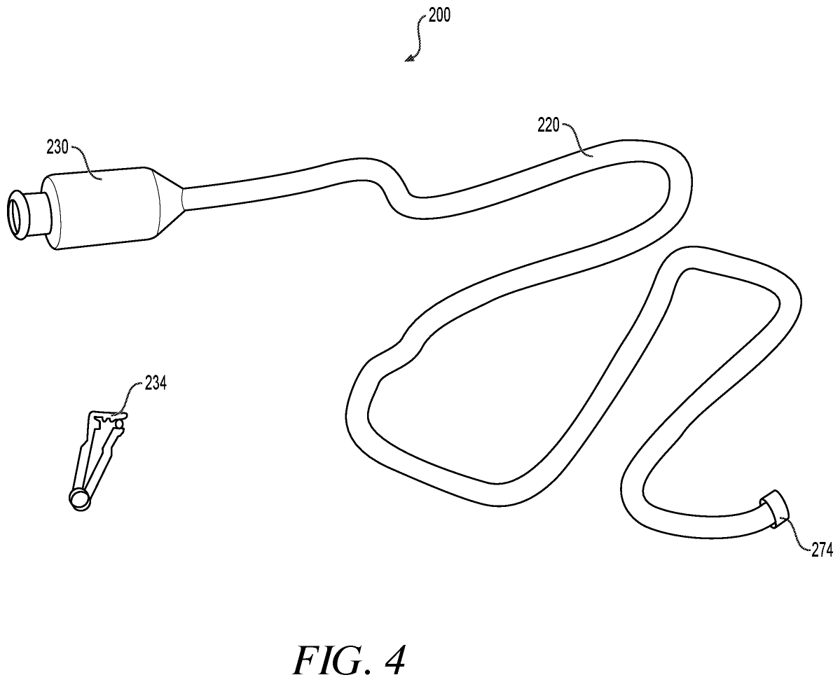

With reference to FIG. 4, an exemplary embodiment of an enema administration device 200 is disclosed. Enema administration device 200 may comprise enema catheter 220, catheter connector 230, and/or insertion tip connector 274. Enema administration device 200 may further include catheter clamp 234 positioned upon enema catheter 220 or included separately (as shown), and configured to block flow through enema catheter 220 when appropriate. In alternative embodiments, clamp 234 may be omitted or may be an adjustable clamp.

In certain embodiments, it is contemplated that catheter connector 230 of device 200 may be configured to fluidly connect with tubing connector 180 of apparatus 100 in a manner substantially similar to the engagement of tubing connector 180 irrigation/medication port 30 of an existing fecal management system 1. Accordingly, as tubing connector 180 may be a male leur lock connector, catheter connector 230 may be a female leur lock connector in preferred embodiments. Additionally, it is contemplated that catheter connector 230 may substantially match or otherwise correspond to tubing connector 180 in color to avoid potential medical provider confusion. Accordingly, it is contemplated that catheter connector 230 may be substantially white and/or translucent in some embodiments; catheter connector 230 may be substantially blue in other embodiments.

Insertion tip connector 274 may be configured to configured to engage with attachment port 284 of insertion tip 280 to allow for a secure fluid connection between enema catheter 220 and insertion tip 280. In some embodiments, insertion tip connector 274 may be a male connector and attachment port 284 may be a female connector; in other embodiments, this may be reversed. Insertion tip connector 274 and attachment port 284 may comprise leur lock connectors or any other suitable connection mechanisms known in the art.

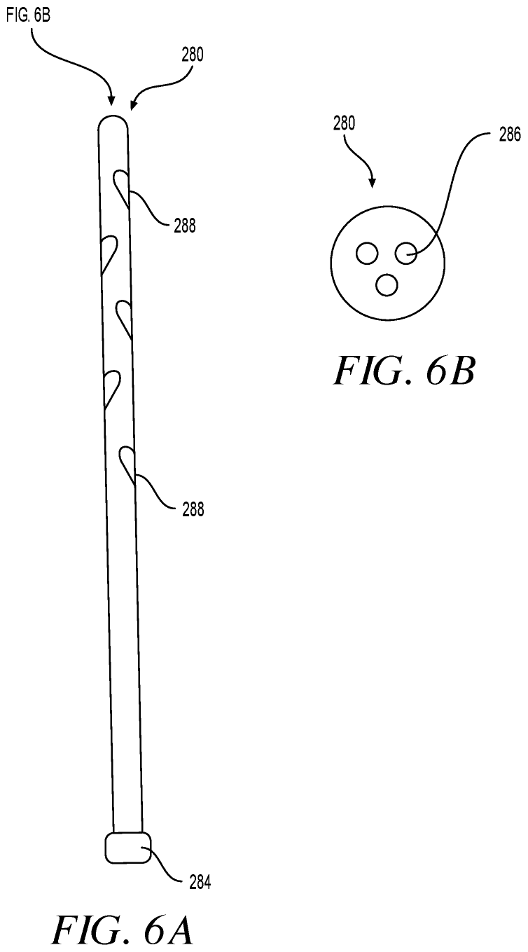

With reference to FIGS. 6A and 6B, an exemplary embodiment of enema insertion tip 280 is disclosed. Insertion tip 280 may include attachment port 284, one or more side openings 288, and one or more top holes 286. In some embodiments, enema insertion tip 280 may be 10-12 inches and/or may substantially comprise silicone. Various embodiments of enema insertion tip 280 may be between 6-18 Fr to accommodate ICU patients (e.g., 15-18 Fr), PICU patients (e.g., 9-14 Fr) depending on the child size, and even NICU patent (e.g., 6- 8 Fr). It is contemplated that enema insertion tip 280 may have one or more insertion markings (not shown) to indicate how much of tip 280 should be rectally inserted. For example, an insertion marking for an infant may be approximately 2.5 cm (1″) from the distal end of tip 280; an insertion marking for a child of 2-3 year old may be approximately 5 cm (2″) from the distal end of tip 280; an insertion marking for a child of 4-10 year old may be approximately 7.5 cm (3″) from the distal end of tip 280; and an insertion marking for a child of 10 years old or older may be approximately 10 cm (4″) from the distal end of tip 280. Insertion markings for adolescent or adult use may be 10 cm (4″) from the distal end of tip 280 or further, depending on the size of the patient.

In some embodiments, one or more side openings 288 may be substantially oval in shape and/or slanted with respect to the central axis of enema insertion tip 280. With reference to FIG. 6B, one or more top holes 286 may be positioned at the end of enema insertion tip 280 opposite from attachment port 284.

In some embodiments of apparatus 200, insertion tip connector 274 may be omitted (not shown in FIG. 4). In such embodiments, it is contemplated that the distal end of enema catheter 220 may be directly inserted into a patient's rectum. In other embodiments, insertion tip 280 may integrally formed or directly connected with enema catheter 220; in such embodiments, insertion tip connector 274 and attachment port 284 may be omitted.

In various embodiments, enema catheter 220 may have an external diameter of 0.8 cm-1.2 cm, more preferably a diameter of 0.6 cm-0.8 cm, or even more preferably a diameter of 0.4 cm-0.6 cm. A reduced diameter may be advantageous with respect to improving patient comfort and ease of anal insertion, particularly in embodiments where insertion tip connector 274 is omitted.

In some embodiments, enema solution providing apparatus 100 and enema administration device 200 may be sold and manufactured as a combined system 100/200. In such embodiments, enema catheter 220 may be integrally formed with tubing 170, or at least with respect to a lower tubing component of tubing 170; and catheter connector 230 and tubing connector 180 may be omitted. Optionally, catheter clamp 234 may also be omitted in such embodiments.

Enema solution providing apparatus 100 and enema administration device 200 may be used in conjunction in a manner somewhat similar to a traditional enema bag 50 to administer an improved enema. During operation of enema system 100/200, a filled drip bag 160 may be provided and positioned above the patient, for example, using an IV pole, hook, hanger, and/or the like. Catheter connector 230 may be attached to tubing connector 180. Enema insertion tip 280 (or the end of enema catheter 220) may be lubricated and then positioned within the patient's rectum. If catheter clamp 234 is being used to block flow through enema catheter 220, it may be released. Adjustable clamp 172 may be actuated to allow enema solution to flow from drip bag 160, through tubing 170, through enema catheter 220, and into the patient's lower gastrointestinal tract. Adjustable clamp 172 may be adjusted (and, in some circumstances, re-adjusted) by a medical provider as appropriate to provide a desired rate of flow. When the administration of enema fluid is complete, tubing 170 and/or enema catheter 220 may be re-clamped. Enema insertion tip 280 (or the end of enema catheter 220) may be removed from the patient and system 100/200 may be discarded.

Consistent with the above disclosures, various enema-related medical kit embodiments are contemplated. In one kit embodiment, some or all fecal management system 1 components may be included along with some or all of the components of an embodiment of enema solution providing apparatus 100.

In another kit embodiment, some or all of the components of an embodiment of enema solution providing apparatus 100 may be included. A leur lock syringe may optionally be additionally included.

In yet another kit embodiment, some or all of the components of an embodiment of enema administration device 200 may be included. One or more insertion tips 280 may also be included. In embodiments where a plurality of insertion tips 280 are included, they may be of a variety of sizes, in length and/or diameters.

In yet another kit embodiment, substantially all components of enema system 100/200 may be provided. That is some or all of the components of an embodiment of enema solution providing apparatus 100 and some or all of the components of an embodiment of enema administration device 200 may be provided. One or more insertion tips 280 may also be provided. A leur lock syringe may optionally be additionally included.

Although the foregoing embodiments have been described in detail by way of illustration and example for purposes of clarity of understanding, it will be readily apparent to those of ordinary skill in the art in light of the description herein that certain changes and modifications may be made thereto without departing from the spirit or scope of the disclosure. It is also to be understood that the terminology used herein is for the purpose of describing particular aspects only, and is not intended to be limiting, since the scope of the present invention will be limited only by claims submitted in an application which claims priority to the instant application.

It is noted that, as used herein, the singular forms “a”, “an”, and “the” include plural referents unless the context clearly dictates otherwise. It is further noted that the claims in an application that claims priority to the instant disclosure may be drafted to exclude any optional element. As such, this statement is intended to serve as antecedent basis for use of such exclusive terminology as “solely,” “only,” and the like in connection with the recitation of claim elements, or use of a “negative” limitation. As will be apparent to those of ordinary skill in the art upon reading this disclosure, each of the individual aspects described and illustrated herein has discrete components and features which may be readily separated from or combined with the features of any of the other several aspects without departing from the scope or spirit of the disclosure. Any recited method can be carried out in the order of events recited or in any other order that is logically possible. Accordingly, the preceding merely provides illustrative examples. It will be appreciated that those of ordinary skill in the art will be able to devise various arrangements which, although not explicitly described or shown herein, embody the principles of the disclosure and are included within its spirit and scope.

Furthermore, all examples and conditional language recited herein are principally intended to aid the reader in understanding the principles of the invention and the concepts contributed by the inventors to furthering the art, and are to be construed without limitation to such specifically recited examples and conditions. Moreover, all statements herein reciting principles and aspects of the invention, as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary configurations shown and described herein.

In this specification, various preferred embodiments have been described with reference to the accompanying drawings. It will be apparent, however, that various other modifications and changes may be made thereto and additional embodiments may be implemented without departing from the broader scope of this disclosure. The specification and drawings are accordingly to be regarded in an illustrative rather than restrictive sense.

We claim:

1. A method of administering an enema to a patient utilizing a fecal management system, comprising:
providing the patient utilizing the fecal management system, the fecal management system including an irrigation port, a catheter, and a collection bag;
providing an enema solution providing apparatus, the enema solution providing apparatus including a bag containing enema solution, tubing, a tubing connector, and an adjustable clamp;
clamping the catheter between the irrigation port and the collection bag;
engaging the tubing connector with the irrigation port;
actuating the adjustable clamp to commence a flow of enema solution from the bag containing enema solution into the patient through the catheter;
completing the flow of enema solution from the bag containing enema solution into the patient;
disengaging the tubing connector from the irrigation port; and
unclamping the catheter.

2. The method of claim 1, further comprising:
actuating the adjustable clamp to adjust the flow, after the step of actuating the adjustable clamp to commence the flow and before the step of completing the flow.

3. The method of claim 2, wherein the enema solution providing apparatus further includes a drip chamber, further comprising:
monitoring the drip chamber prior to the step of actuating the adjustable clamp to adjust the flow.

4. The method of claim 2, wherein the step of actuating the adjustable clamp to adjust the flow comprises:
receiving feedback from the patient; and
actuating the adjustable clamp to adjust the flow in response to the feedback.

5. The method of claim 2, wherein:
the adjustable clamp is a roller clamp; and
the step of actuating the adjustable clamp to adjust the flow includes rolling a roller of the roller clamp.

6. The method of claim 1, wherein the step of actuating the adjustable clamp to commence the flow of enema solution further comprises:
actuating the adjustable clamp to an intermediate position between blocking flow and allowing maximum flow.

7. The method of claim 2, wherein the step of actuating the adjustable clamp to adjust the flow further comprises:
actuating the adjustable clamp to an intermediate position between blocking flow and allowing maximum flow.

8. The method of claim 1, wherein the step of engaging the tubing connector with the irrigation port further comprises:
engaging a female leur lock connector of the irrigation port with a male leur lock connector of the tubing connector.

9. The method of claim 1, wherein the step of completing the flow of enema solution from the bag containing enema solution into the patient further comprises:
actuating the adjustable clamp to block the flow.

10. The method of claim 1, wherein the step of providing an enema solution providing apparatus further comprises:
providing the enema solution providing apparatus with a color of the tubing connector that corresponds to the color of the irrigation port.

11. A kit for providing an enema through a fecal management system, the kit comprising:
an adjustable clamp;
tubing, the tubing configured to attach to a drip bag at a first end; and
a tubing connector, the tubing connector provided at a second end of the tubing.

12. The kit of claim 11, wherein the tubing connector comprises a male leur lock connector.

13. The kit of claim 12, wherein the adjustable clamp is a roller clamp.

14. The kit of claim 11, further comprising:
a drip chamber,
wherein the drip chamber is provided along the path of the tubing.

15. The kit of claim 11, further comprising the drip bag.

16. The kit of claim 16, wherein the drip bag is pre-filled with an enema solution

17. The kit of claim 11, further comprising a fecal management system.

18. An enema solution providing apparatus, comprising:
a drip bag, the drip bag containing an enema solution;
tubing, the tubing fluidly connected to the drip bag at a first end;
an adjustable clamp, the adjustable clamp positioned along the tubing; and
a tubing connector, the tubing connector attached to a second end of the tubing;
wherein:
the tubing connector comprises a male leur lock connector; and
the adjustable clamp has at least first position configured to block a flow of the enema solution through the tubing, a second position configured to allow a maximum flow of the enema solution through the tubing, and a third position configured to allow an intermediate flow of the enema solution through the tubing.

19. The enema solution providing apparatus of claim 18, wherein the adjustable clamp is a roller clamp.

20. The enema solution providing apparatus of claim 19, further comprising:
a drip chamber,
wherein the drip chamber is provided along the path of the tubing.
